(19) United States
(12) Patent Application Publication
(10) Pub. No.: US 2026/0306529 A1
(43) Pub. Date: Oct. 8, 2026

(54) COMPOSITION COMPRISING HOMOGENEOUS POLYSACCHARIDE OR DERIVATIVE THEREOF AND METHOD OF USING THE SAME TO PREVENT AND/OR TREAT BONE LOSS

(71) Applicant: Chang CHEN

(72) Inventor: Chang CHEN

(21) Appl. No.: 18/880,518

(22) PCT Filed: Jul. 13, 2023
(86) PCT No.: PCT/IB2023/000414

§ 371 Date: Dec. 31, 2024

(51) Int. Cl. A61K 31/715; A61K 31/737; A61K 36/815; A61P 19/08

(52) U.S. Cl. CPC A61K 31/715; A61K 31/737; A61K 36/815; A61P 19/08; A61K 2236/331; A61K 2236/51; A61K 2236/55

(57) ABSTRACT

A composition and a method are provided for improving formation, mass and/or strength of bones, and/or treating or preventing bone loss such as age-related bone loss in a subject in need thereof. Such a composition comprising a homogenous polysaccharide, or a pharmaceutically acceptable ester or salt thereof, or a pharmaceutically acceptable solvate thereof, or any combination thereof, and a pharmaceutically acceptable excipient. The homogeneous polysaccharide consists essentially of arabinose, galactose, rhamnose, and galacturonic acid as monomer units. Such a method comprises administrating an effective amount of such a composition into a subject in need thereof.

### PRIORITY CLAIM AND CROSS-REFERENCE

This application is a national stage application of International Application No. PCT/IB2023/00414, filed Jul. 13, 2023, which claims the benefit of U.S. Provisional Application No. 63/389,082, filed Jul. 14, 2022, which applications are expressly incorporated by reference herein in their entirety.

### FIELD OF THE INVENTION

The disclosure relates to generally a composition having pharmaceutical or functional properties. More particularly, the disclosed subject matter relates to a Lycium barbarum L. extract, a resulting composition comprising polysaccharide or a derivative thereof, and a method of using the same, for example, as a pharmaceutical composition, a functional composition, and/or a dietary supplement.

### BACKGROUND

In a recent study on the epidemiology of osteoporosis in the European Union, the prevalence of osteoporosis showed European women aged 50-55 years and 47% in women of 80 years or older. For European men of the same age groups, the prevalence was estimated at 2.5% and 16%, respectively. The loss of bone mass is not limited to postmenopausal women, there is increasing attention being paid to osteoporosis in older men and this number is expected to steadily increase with an aging population. It has been suggested that for one in three women or one in five men over 50 years old, bone loss leads to a rapid increase in fracture risk and mortality in the elderly. At present, more than 200 million people suffer from osteoporosis around the world. Osteoporosis is characterized by a reduction in bone mass and density coupled with disrupted bone microarchitecture. Therefore, senile osteoporosis is an emerging medical and socioeconomic threat due to its high prevalence, high disability rate and high fatality rate.

Senile osteoporosis is predominantly associated with reduced bone formation, and low bone turnover, and age-related bone loss is caused by impaired bone formation compared to bone resorption. Bone morphogenic protein (BMP) signaling has fundamental roles in both skeletal development and bone homeostasis. BMPs are members of the transforming growth factor beta (TGF-β) superfamily and signal by binding and assembling type I and type II transmembrane serine/threonine receptor kinases. After ligand-induced assembly of two type I and two type II receptors, the constitutively active type II receptor kinases phosphorylate and activate the type I receptors, which in turn phosphorylate and leads to signal transduced through either Smads or MAPKs, which further activates the transcription of specific target genes involved in osteoblastic differentiation and bone formation. Noggin is well-known as a potent inhibitor of BMPs and thus of osteogenic function in vivo and in vitro, noggin bind to BMPs with high adhesion force, and then prevent them from binding to BMP receptors. BMPs have essential roles at many steps in bone development. Studies indicate that BMP-2 has a unique role in postnatal bone formation. Human recombinant bone morphogenetic protein-2 (rhBMP2) as a kind of potent osteogenic growth factors approved by Food and Drug Administration, has been widely used for bone tissue engineering. Therefore, the discovery of natural products that have the mimetic effects of BMP-2 in osteogenic function has great importance and significance.

### SUMMARY

The present disclosure provides a composition and a method for improving formation, mass and/or strength of bones, and/or treating or preventing bone loss in a subject in need thereof.

In one aspect, the present disclosure provides a method for improving bone formation, bone mass and/or bone strength. Such a method can be a method for treating or preventing bone loss such as age-related bone loss in a subject in need thereof. In accordance with some embodiments, such a method comprises administrating an effective amount of a composition comprising a homogenous polysaccharide, or a pharmaceutically acceptable ester or salt thereof, or a pharmaceutically acceptable solvate thereof, or any combination thereof, and a pharmaceutically acceptable excipient into a subject in need thereof. The homogeneous polysaccharide consists essentially of arabinose, galactose, rhamnose, and galacturonic acid as monomer units or moieties.

In some embodiments, the subject is a mammal, preferably a human subject, which can be a healthy human, or an aging adult, or any other adult having bone loss.

The composition can be a pharmaceutical composition, a functional composition, and/or a dietary supplement. In some embodiments, the composition is orally administrated or injected into stomach. The composition may be in a tablet form or a liquid form. For example, in some embodiments, the composition is a pharmaceutical composition in a tablet form, which can be orally administrated. In some embodiments, the composition may be a functional composition and in a dry powder form. The composition may be also formulated in a sports drink or a snack bar form.

The homogeneous polysaccharide may have a molecular weight in a range of from about 10 kDa to about 150 kDa, for example, from about 10 kDa to about 100 kDa, from about 10 kDa to 90 kDa, from about 10 kDa to 60 kDa, or any other suitable ranges. In the homogeneous polysaccharide, the molar ratio of monomer units of arabinose, galactose, rhamnose, and galacturonic acid is in a range of from 30-70:20-60:0.1-10:0.1-10. For example, the homogeneous polysaccharide used in the experiments of the present disclosure, called LBP1C-2, has a ratio of monomer units of arabinose, galactose, rhamnose, and galacturonic acid being 49.9:33.6:8.0:8.5. Its molecular weight may be about a specific value or a narrow range in a range of from about 10 kDa to about 150 kDa. Each polysaccharide obtained is homogenous with uniform or a narrow molecular weight distribution.

In some embodiments, the homogeneous polysaccharide as described herein is the only polysaccharide in the composition.

In some other embodiments, the composition further comprises additional polysaccharide isolated from a Lycium barbarum extract. The homogenous polysaccharide is at least 15% of all polysaccharides in the composition. All the polysaccharides may be isolated from Lycium barbarum. For example, the polysaccharides used in the present disclosure are Lycium barbarum polysaccharides (called LBP).

In some embodiments, the composition further optionally comprises one or more compounds selected from the group consisting of flavone, carotenoid, polyphenol, pigment, or any combination isolated from a Lycium barbarum extract.

In some embodiments, the composition comprises a chemical modified derivative of the homogenous polysaccharide as described herein. For example, such a derivative is a pharmaceutically acceptable ester or salt thereof. The pharmaceutically acceptable ester or salt thereof is a sulfate ester derivative of the homogeneous polysaccharide or called sulfated polysaccharide.

The excipient may be a solvent, a co-solvent, a coloring agent, a preservative, an antimicrobial agent, a filler, a binder, a disintegrating agent, a lubricant, a surfactant, an emulsifying agent, a suspending agent, or any combination thereof.

The composition may be administrated in any suitable amount. For example, in some embodiments, the dose of the effective amount of the composition (by the amount of the homogenous polysaccharide as described herein) is in a range of from 10 mg/Kg to 500 mg/Kg based on a total daily weight of the homogeneous polysaccharide/a body weight of the subject on daily basis. The composition may be administrated once daily, twice daily, or more than twice per day.

In another aspect, the present disclosure provides a composition (as described herein) for improving bone formation, bone mass and/or bone strength. The composition can be used for treating or preventing bone loss such as age-related bone loss in a subject in need thereof. Such a composition comprises an effective amount of a homogenous polysaccharide or a derivative thereof and a pharmaceutically acceptable excipient. The homogeneous polysaccharide consists essentially of arabinose, galactose, rhamnose, and galacturonic acid as monomer units or moieties.

As described herein, the composition is a pharmaceutical composition, a functional composition, and/or a dietary supplement. For example, the composition is an oral composition and/or is in a tablet form in some embodiments.

The excipient may be a solvent, a co-solvent, a coloring agent, a preservative, an antimicrobial agent, a filler, a binder, a disintegrate, a lubricant, a surfactant, an emulsifying agent, a suspending agent, or any combination thereof.

The homogeneous polysaccharide has a molecular weight in a range of from about 10 kDa to about 150 kDa, for example, from about 10 kDa to about 100 kDa, from about 10 kDa to about 80 kDa, from about 10 kDa to about 60 kDa, or any other suitable ranges. The homogeneous polysaccharide has a molar ratio of monomer units of arabinose, galactose, rhamnose, and galacturonic acid in a range of from 30-70:20-60:0.1-10:0.1-10. For example, in some embodiments, the homogeneous polysaccharide has a ratio of monomer units of arabinose, galactose, rhamnose, and galacturonic acid being 49.9:33.6:8.0:8.5. Its molecular weight may be about a specific value or a narrow range in a range of from about 10 kDa to about 150 kDa. Each polysaccharide obtained is homogenous with uniform or a narrow molecular weight distribution.

In some embodiments, the homogeneous polysaccharide as described herein is the only polysaccharide in the composition. In some other embodiments, the composition further comprises additional polysaccharide isolated from a Lycium barbarum extract, and/or the homogenous polysaccharide is at least 15% of all polysaccharides in the composition.

In some embodiments, the composition may optionally include flavone, carotenoid, polyphenol, pigment, or any combination, which is isolated from a Lycium barbarum extract. In other some embodiments, the composition does not include flavone, carotenoid, or polyphenol isolated from a Lycium barbarum extract.

In some embodiments, the composition comprises a chemical modified derivative of the homogenous polysaccharide as described herein. For example, such a derivative is a pharmaceutically acceptable ester or salt thereof. The pharmaceutically acceptable ester or salt thereof is a sulfate ester derivative of the homogeneous polysaccharide or called sulfated polysaccharide.

The present disclosure also provides the use of the homogenous polysaccharide or its derivative as described herein for the manufacture of a medicament for the treatment of any of these medical conditions as described herein.

In another aspect, the present disclosure provides a method of making the composition or the homogenous polysaccharide as described herein. Such a method may include preparing or isolating the homogenous polysaccharide. The method may further comprise mixing the excipient and the homogenous polysaccharide. The method may include chemically modifying such as sulfating the homogenous polysaccharide.

### BRIEF DESCRIPTION OF THE DRAWINGS

The present disclosure is best understood from the following detailed description when read in conjunction with the accompanying drawings. It is emphasized that, according to common practice, the various features of the drawings are not necessarily to scale. On the contrary, the dimensions of the various features are arbitrarily expanded or reduced for clarity. Like reference numerals denote like features throughout specification and drawings.

FIG. 1 shows the trabecular structural of the mice in the control group, and the mice in the experimental group administrated with the Lycium barbarum water extract (LBE), Lycium barbarum crude polysaccharides (LBP) and Lycium barbarum homogeneous polysaccharide (LBP1C-2).

FIG. 2 shows the bone mineral density (BMD) values (FIG. 2A), trabecular bone volume to total volume (BV/TV) ratio (FIG. 2B), trabecular number (Tb.N) (FIG. 2C), trabecular thickness (Tb.Th) (FIG. 2D), and trabecular separation (Tb.Sp) (FIG. 2E) of the mice in the control group, and the mice in the experimental group administrated with LBE, LBP and LBP1C-2.

FIG. 3 shows the maximum load a (as assessed by the maximum load in the femur using the three-point bending test) of the mice in the control group, and the mice in the experimental group administrated with LBE, LBP and LBP1C-2.

FIG. 4 shows the bone formation rate of the mice in the control group, and the mice in the experimental group administrated with LBE, LBP and LBP1C-2. The abbreviation “BFR/BS” represents bone formation rate per bone surface.

FIG. 5 shows the mineral apposition rate (MAR) of the mice in the control group, and the mice in the experimental group administrated with LBE, LBP and LBP1C-2.

FIG. 6 shows the osteocalcin content in the serum of the mice in the control group, and the mice in the experimental group administrated with LBE, LBP and LBP1C-2.

FIG. 7 shows the procollagen I N-terminal peptide (PINP) content in serum of the mice in the control group, and the mice in the experimental group administrated with LBE, LBP and LBP1C-2.

FIG. 8 shows the osteoblast proliferation in human mesenchymal stem cells (hMSCs) upon treatment with LBE, LBP, LBP1C-2 and the hMSCs in the control group in a colony-forming unit (CFU) assay.

FIG. 9 shows the expression level of osteocalcin in hMSCs treatment with LBE, LBP, LBP1C-2 and the hMSCs in the control group.

FIG. 10 shows the alkaline phosphatase (ALP) enzyme activity in hMSCs treatment with LBE, LBP, LBP1C-2 and the hMSCs in the control group.

FIG. 11 shows the expression levels of bone formation related genes in the femur of the mice in the control group and the mice in the experimental group administrated with LBP1C-2.

FIG. 12 shows the expression levels of bone resorption related genes in the femur of the mice in the control group and the mice in the experimental group administrated with LBP1C-2.

FIGS. 13-15 show the expression levels of bone formation related genes, Runx2, Col1α and Bglap in pre-osteoblasts treated with LBP1C-2 and those in the control group after the Bmpr1a and Bmpr2 genes were disturbed.

FIGS. 16-17 show the direct interaction of LBP1C-2 with BMPRIA and BMPRII by the results of surface plasmon resonance (SPR) in the control group and the experimental group with LBP1C-2.

FIGS. 18-19 show the direct interaction of LBP1C-2 with BMPRIA and BMPRII by the results of fluorescence-based thermal shift assay in the control group and the experimental group with LBP1C-2.

FIGS. 20A-20B show the phosphorylation level of Smad1/5/8 in hMSCs treatment with LBP1C-2 and the hMSCs in the control group. FIG. 20A shows the expression levels of the phosphorylation level of Smad1/5/8, Smad1/5/8 and GAPDH. FIG. 20B shows the western blot analyses of the level of the phosphorylation level of Smad1/5/8, treated with or without LBP1C-2. GAPDH was used as the loading control.

FIG. 21 shows the direct interaction of LBP1C-2 with Noggin by the results of surface plasmon resonance (SPR) in the control group and the experimental group with LBP1C-2.

FIG. 22 shows the release level of bone morphogenic protein 2 (BMP2) in hMSCs treatment with LBP1C-2 and the hMSCs in the control group.

FIGS. 23A-23B show the levels of the early osteogenic markers of bone formation, RUNX2 (FIG. 23A) and SP7 (FIG. 23B) in hMSCs treatment with LBP1C-2, S-LBP1C-2 (sulfated LBP1C-2) and the hMSCs in the control group.

FIG. 24A shows the alkaline phosphatase (ALP) staining (7th day) in hMSCs treatment with LBP1C-2, S-LBP1C-2 and the hMSCs in the control group.

FIG. 24 B shows ALP enzyme activity in hMSCs treatment with LBP1C-2, S-LBP1C-2 and the hMSCs in the control group.

FIG. 25A shows alizarin red S staining (21^{st} day) in hMSCs treatment with LBP1C-2, S-LBP1C-2 and the hMSCs in the control group.

FIG. 25 B shows the expression level of osteocalcin in hMSCs treatment with LBP1C-2, S-LBP1C-2 and the hMSCs in the control group.

### DETAILED DESCRIPTION

In the present disclosure the singular forms “a,” “an,” and “the” include the plural reference, and reference to a particular numerical value includes at least that particular value, unless the context clearly indicates otherwise. Thus, for example, a reference to “an additive” is a reference to one or more of such compounds and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is a human. In another embodiment, the subject is a human adult.

As used herein, the term “agent” refers to any molecule, compound, methodology and/or substance for use in the prevention, treatment, management and/or diagnosis of a disease or condition. As used herein, the term “effective amount” refers to the amount of a therapy that is sufficient to result in the prevention of the development, recurrence, or onset of a disease or condition, and one or more symptoms thereof, to enhance or improve the prophylactic effect(s) of another therapy, reduce the severity, the duration of a disease or condition, ameliorate one or more symptoms of a disease or condition, prevent the advancement of a disease or condition, cause regression of a disease or condition, and/or enhance or improve the therapeutic effect(s) of another therapy. The disease or condition (e.g., medical condition) is related to bone loss or condition associated with bone formation, bone mass, and/or bone strength.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “therapeutic agent” refers to any molecule, compound, and/or substance that is used for the purpose of treating and/or managing a disease or disorder.

As used herein, the terms “therapies” and “therapy” can refer to any method(s), composition(s), and/or agent(s) that can be used in the prevention, treatment and/or management of a disease or condition, or one or more symptoms thereof. In certain embodiments, the terms “therapy” and “therapies” refer to small molecule therapy.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, such as cancer, and/or the amelioration of one or more symptoms thereof resulting from the administration of one or more therapies.

As used herein, the term “excipient” refers to an inactive substance that serves as the vehicle or medium for a drug or other active substance. Examples of a suitable excipient include, but are not limited to, a solvent, a co-solvent, a coloring agent, a preservative, an antimicrobial agent, a filler, a binder, a disintegrate, a lubricant, a surfactant, an emulsifying agent, a suspending agent, or any combination thereof.

As used herein, the term “monomer unit” or “monomer units” in a polysaccharide refers to monosaccharide base units or single sugar molecule units or other base chemical units. These units are understood as moieties of monosaccharides and linked together through-O-bonds through condensation reactions among hydroxyl groups.

The molecular weight of is measured using gel permeation chromatography (GPC). GPC is an analytical technique that separates molecules in polymers by size and provides the molecular weight or molecular weight distribution of a material. The homogeneous polysaccharide as described herein contain only one peak in GPC with one uniform molecular weight. A homogenous polysaccharide or its derivative is provided and used in the present disclosure. Because of its uniformity in molecular weight distribution (i.e. with a polydispersity index being 1 or close to 1), its weight-averaged molecular weight (Mw), its number averaged molecular weight (Mn), or its peak molecular weight (Mp) are the same, or about the same, or very close to each other. Unless expressly indicated otherwise, the molecular weight values and ranges described herein can be Mw or Mn. In some embodiments, the molecular weight values and ranges described herein are number averaged molecular weight (Mn).

Lycium barbarum (Gouqi or wolfberry) can be used as a Chinese medicine and healthy eatable food in China and other countries, and it belongs to the genus Lycium of the family solanaceae. L. barbarum is listed as a homologous species for possible use in medicine and food in China. The fruits of L. barbarum have effects on anti-aging and anti-fatigue. L. barbarum has been used to nourish the liver and kidney, improve vision and possibly against diseases. However, until now, little is known about the molecular mechanism of the function of L. barbarum, and the effective composition is not clear.

The inventors have discovered the function of L. barbarum in muscle. There are multitudinous components in L. barbarum, such as polysaccharides, flavonoids, betaine, taurine, vitamin, fatty acids and so on. Polysaccharides of L. barbarum as the main active ingredient have biological activities including antioxidant, antivirus, neuroprotective properties, acute liver injury and immunomodulatory activities.

In recent works, using a unique isolation method, the inventors extracted and isolated total water extracts, i.e., the L. barbarum extract (LBE), and crude polysaccharides (LBP) from fruits of L. barbarum. A homogeneous polysaccharide named LBP1C-2 (for example, with a yield of 0.02% of dried fruits) was purified from LBP. LBP1C-2 has been found as a pectin. However, whether LBP1C-2 has effect on bone remodeling is unknown.

Proliferation, differentiation and mineralization in osteroblasts promoted by a sample of L. barbarum polysaccharides in vitro have been founded. The polysaccharides used were extracted from fruit of L. barbarum, and are composed of six monosaccharides, including galactose, glucose, rhamnose, arabinose, mannose, and xylose. The in vitro experiments were conducted using human mesenchymal stem cells (hMSCs).

However, whether L. barbarum supplement improves aging induced bone loss, what the main active substance is, and related mechanism and target have not been determined.

In the present disclosure, the inventors examined the outcome of LBE, LBP and LBP1C-2 on natural aging mice with an identification of the target and mechanism. LBP1C-2, a homogeneous polysaccharide from L. barbarum extract, was found as an active component of the LBE. The main polysaccharide ingredient LBP1C-2 directly binds BMPRIA and BMPRII to promote phosphorylation of Smads to enhance osteogenic differentiation and mineralization, LBP1C-2 was also directly bound to noggin and inhibited noggin interaction with BMPs to increase bone formation. This study also provided a scientific and molecular base for explaining the efficacy of L. barbarum extract composition and to found scientific evidence for its use as natural dietary or product supplement to treat the age-related bone loss in the future.

The present disclosure provides a composition and a method for improving bone formation, bone mass and/or bone strength. Such a method can be a method for treating or preventing bone loss such as age-related bone loss in a subject in need thereof.

In accordance with some embodiments, such a composition comprises a homogenous polysaccharide, or a pharmaceutically acceptable ester or salt thereof, or a pharmaceutically acceptable solvate thereof, or any combination thereof, and a pharmaceutically acceptable excipient into a subject in need thereof. The homogeneous polysaccharide consists essentially of arabinose, galactose, rhamnose, and galacturonic acid as monomer units or moieties. Such a method comprises administrating an effective amount of a composition comprising the homogenous polysaccharide or its derivative thereof into a subject in need thereof.

In some embodiments, the subject is a mammal, preferably a human subject, which can be a healthy human, or an aging adult, or an adult having bone loss.

The composition can be a pharmaceutical composition, a functional composition, and/or a dietary supplement. In some embodiments, the composition is orally administrated or injected into stomach. The composition may be in a tablet form or a liquid form. For example, in some embodiments, the composition is a pharmaceutical composition in a tablet form, which can be orally administrated. In some embodiments, the homogenous polysaccharide or a derivative thereof described herein is a therapeutic agent. In some embodiments, the composition may be a functional composition and in a dry powder form. The composition may be also formulated in a sports drink or a snack bar form.

The homogeneous polysaccharide has a molecular weight in a range of from about 10 kDa to about 150 kDa, for example, from about 10 kDa to about 100 kDa, from about 10 kDa to about 80 kDa, from about 10 kDa to about 60 kDa, or any other suitable ranges. The homogeneous polysaccharide has a molar ratio of monomer units of arabinose, galactose, rhamnose, and galacturonic acid in a range of from 30-70:20-60:0.1-10:0.1-10. For example, in some embodiments, the homogeneous polysaccharide has a ratio of monomer units of arabinose, galactose, rhamnose, and galacturonic acid being 49.9:33.6:8.0:8.5. Its molecular weight may be a specific value or in a narrow range in a range of from about 10 kDa to about 150 kDa. Such a molecular weight value might be either weight averaged molecular weight (Mw) or number averaged molecular weight (Mn). The values of Mw and Mn may be close to each other because the polysaccharide is homogeneous, with a very narrow molecular weight distribution. The polydispersity (PD) index, which is the ratio of Mw to Mn, may be in a range of from about 1 to about 1.3, from about 1 to about 1.2, from about 1 to about 1.1. In some embodiments, the PD index is close to 1. Because the molecular weight of polysaccharides in Lycium barbarum berries as raw materials may vary due to factors such as growth environment and harvesting season. So the homogenous polysaccharides obtained may have varied molecular weight. However, the polysaccharide obtained in each batch is homogenous, namely, with its molecular weight being uniform or having a narrow distribution. The molecular weight can be controlled through controlling the quality of the raw materials, for example, by having the same growth environment and the growth time before harvested.

In some embodiments, the homogeneous polysaccharide as described herein is the only polysaccharide in the composition.

In some embodiments, the homogeneous polysaccharide is LBP1C-2 or its derivative such as sulfated LBP1C-2 as described herein.

In some other embodiments, in addition to LBP1C-2, the composition further comprises additional polysaccharide isolated from a Lycium barbarum extract. The homogenous polysaccharide is at least 15% of all polysaccharides in the composition. All the polysaccharides may be isolated from Lycium barbarum. For example, the polysaccharides used in the present disclosure are Lycium barbarum polysaccharides (called LBP).

In some embodiments, the composition further optionally comprises one or more compounds, which are selected from the group consisting of flavone, carotenoid, polyphenol, pigment, or any combination isolated from a Lycium barbarum extract.

Lycium barbarum may be extracted using water to provide a Lycium barbarum extract (LBE), which can be in a dry powder form. The extract can be also dissolved in water and then further purified or separated through a fractional purification method. Polysaccharides can be obtained. The polysaccharides are further separated through fractional purification and freezing drying to obtain one or more homogenous polysaccharides in dry powder form.

In some embodiments, the composition comprises a Lycium barbarum extract (LBE). Such a Lycium barbarum extract comprises polysaccharide (or called Lycium barbarum polysaccharide), Lycium barbarum flavone, carotenoid, polyphenols and Lycium barbarum pigment. Each of these ingredients may have only one, or two or more of the same type. For example, the composition may include two or more types of polysaccharides, two or more types of Lycium barbarum flavones, two or more types of carotenoids, two or more types of polyphenols, and/or two or more types of Lycium barbarum pigments.

In some embodiments, in the Lycium barbarum extract (LBE), polysaccharide is present in a range of from about 10.0 wt. % to about 70.0 wt. % (e.g., about 50 wt. % to about 70 wt. %), Lycium barbarum flavone is a range of from about 0.1 wt. % to about 5.0 wt. %, carotenoid is a range of from about 0.1 wt. % to about 3.0 wt. %, polyphenol is a range of from about 0.1 wt. % to about 8.0 wt. %, and Lycium barbarum pigment is a range of from about 0.1 wt. % to about 8.0 wt. %, based on the total dry weight of the extract. In some embodiments, the polysaccharide or polysaccharides are more preferably from about 50.0 wt. % to about 70.0 wt. %. The dry weight is equivalent weight corresponding to the extract in a dry powder (without water). The extract may contain other residues of a very small amount. The extract in the form of a dry powder can be mixed with water to provide an extract in the form of an aqueous solution having a selected concentration as described herein.

In some embodiments, the Lycium barbarum extract (LBE) is in a powder form. In some embodiments, the Lycium barbarum extract is dissolved into a solvent such as water to provide an aqueous liquid having a concentration, for example, in a range of from 0.1 g/mL to 5 g/mL. In the experiments, in the LBE used, the polysaccharides are in a range from about 50.0 wt. % to about 70.0 wt. % in the dry powder form of the LBE.

Lycium barbarum polysaccharide or polysaccharides (LBP), which is in a powder form, can be further purified from the LBE in the powder form. For example, the LBE can be dissolved in water and then separated by going through separation columns. Lycium barbarum polysaccharide (LBP) may include different polysaccharides, which can be further separated.

From the LBE and/or LBP, a homogenous polysaccharide LBP1C-2 is isolated. Based on high performance gel permeation chromatography (HPGPC) analysis, LBP1C-2 showed a single and symmetrical peak, which indicated that it is a homogeneous polysaccharide. According to the sugar composition analysis, LBP1C-2 is composed of arabinose (Ara), galactose (Gal), rhamnose (Rha), and galacturonic acid in a ratio of 49.9:33.6:8.0:8.5. The structure of LBP1C-2 includes a backbone of alternate 1, 2-linked α-Rhap and 1, 4-linked α-GalpA, with branches of terminal (T)-, 1, 3-, 1, 6- and 1, 3, 6-linked β-Galp, T-, 1,5- and 1, 3, 5-linked α-Araf and T-linked β-Rhap substituted at C-4 of 1, 2, 4-linked α-Rhap.

The structure of LBP1C-2 is illustrated in Schemes 1, 2 and 3, which show the same structures in three different formats.

Referring to Schemes 1-3, LBP1C-2 consists of Ara, Gal, Rha, GalA and has a molar ratio of 49.9:33.6:8.0:8.5. Structural analysis showed that LBP1C-2 is mainly composed of 1, 2-α-Rha and 1, 4-α-GalA as the main chains, and the branches include T-α-Ara, 1,5-α-Ara, T-β-Rha, T-β-Gal, 1,3-β-Gal, 1,6-β-Gal and 1, 3, 6-β-Gal, which are attached at the C-4 position of the 1, 2, 4-α-Rha backbone sugar residue. The repeat unit of LBP1C-2 contains the structural parts shown in Scheme 3, and contains a backbone (composed of 1, 2-α-Rha, 1, 2, 4-α-Rha and 1, 4-α-GalA) and three kinds of branches including R1, R2 and R3.

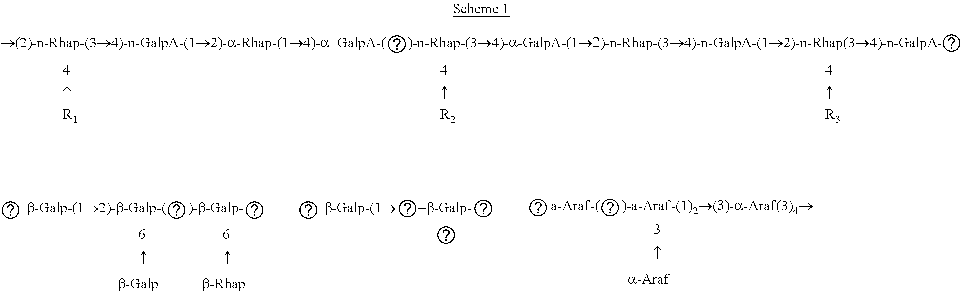

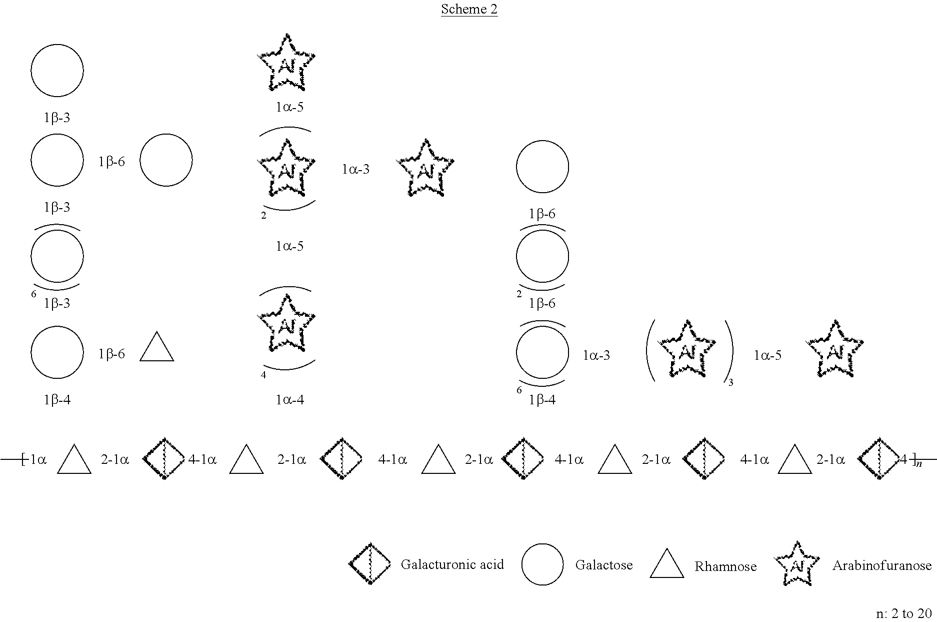

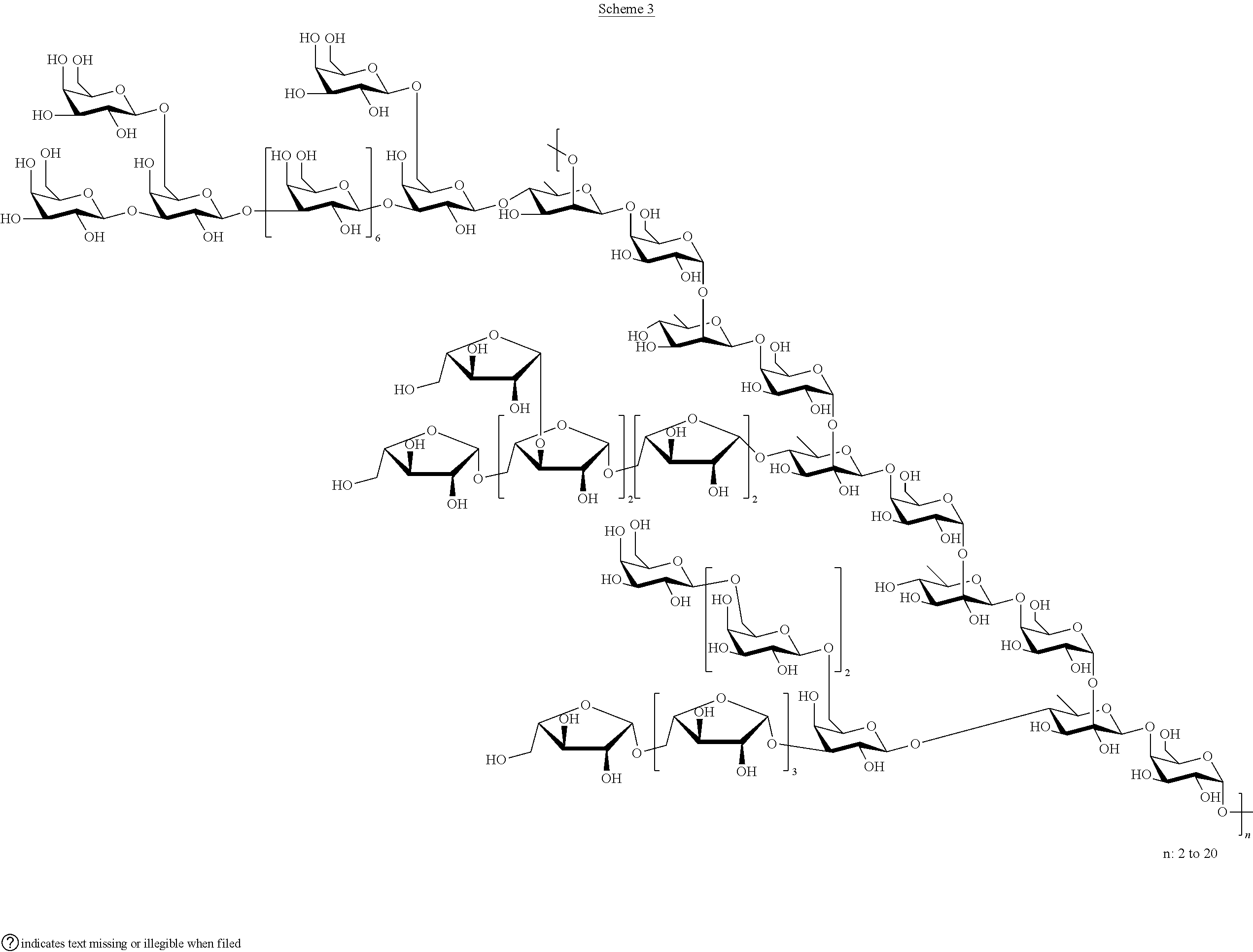

In Schemes 1-3, n is in a range of from 2 to 20. The molecular weight is proportional to the value of n. For example, when a sample shows a molecular weight of about 13.2 kDa, n is about 2. When a sample shows a molecular weight of about 99.8 kDa, n is about 13.

The inventors of the present disclosure have proved that the homogenous polysaccharide LBP1C-2 is the polysaccharide or the active ingredient in the LBE or LBP having efficacy for improving bone formation, bone mass and/or bone strength. The homogenous polysaccharide LBP1C-2 is an active ingredient for treating or preventing bone loss such as age-related bone loss such as age-related bone loss in a subject in need thereof.

In some embodiments, the composition comprises a chemical modified derivative of the homogenous polysaccharide as described herein. For example, such a derivative is a pharmaceutically acceptable ester or salt thereof. The pharmaceutically acceptable ester or salt thereof is a sulfate ester derivative of the homogeneous polysaccharide or called sulfated polysaccharide. In the synthesis of the sulfated homogenous polysaccharide, hydroxyl groups in the homogenous polysaccharide is reacted with a modifying agent such as chlorosulfonic acid to form —O—SO₃H group. The molecular weight after modification remains the same as those described for the homogenous polysaccharide. The sulfate ester derivative of the homogeneous polysaccharide has a degree of sulfate substitution in a range of from 0.5 to 0.9, for example, in a range of from 0.6 to 0.8. The degree of substitution represents the number of substitution groups on a sugar unit. For example, a degree of substitution of 0.74 indicates that the number of sulfated substituents on each hexose or pentose unit is 0.74. The sulfate substitution can binding with proteins and improve the biological activities.

The composition can be a pharmaceutical composition, a functional composition, and/or a dietary supplement. For example, the composition is a pharmaceutical composition, which can be orally administrated. In the embodiment of the present invention, the aqueous extract of Lycium barbarum is administered by gavage or orally, but it is not limited thereto. Any form of administration of having the composition into stomach may be suitable.

The excipient may be a solvent (such as water or an aqueous based solvent), a co-solvent, a coloring agent, a preservative, an antimicrobial agent, a filler, a binder, a disintegrating agent, a lubricant, a surfactant, an emulsifying agent, a suspending agent, or any combination thereof.

The composition may be administrated in any suitable amount. For example, in some embodiments, the dose of the effective amount of the composition (by the amount of the homogenous polysaccharide as described herein) is in a range of from 10 mg/Kg to 500 mg/Kg based on a total daily weight of the homogeneous polysaccharide/a body weight of the subject on daily basis. In some embodiments, the dosage of the Lycium barbarum extract (LBE) or LBP or LBP1C-2 is in a range of from 4 mg/Kg to 70 mg/Kg (the daily dry weight of LBP1C-2 in total/the body weight of subject such as a human being) per day, for example, from 10 mg/Kg to 70 mg/Kg, from 10 mg/Kg to 60 mg/Kg, from 20 mg/Kg to 70 mg/Kg, from 20 mg/Kg to 60 mg/Kg, from 20 mg/Kg to 50 mg/Kg, or any other suitable range. The composition may be administrated once daily, twice daily, or more than twice per day.

In some embodiments of the present invention, the dosage of the Lycium barbarum extract (LBE) or LBP or LBP1C-2 is 40 mg/Kg (the daily dry weight of LBP1C-2 in total/the body weight of animals such as mice) per day. The total amount per day can be dosed once or from two or more times per day. In the Experiments described herein, the mice were administrated with LBE, LBP or LBP1C-2 once daily.

The descriptions on the dosage and the administration methods described herein are also applicable to the derivative of the homogenous polysaccharide LBP1C-2.

In some embodiments, the compositions may be administrated with drink, food, or related ingredients. The water extract of Lycium barbarum provided by the invention is homologous in medicine and food, and can be used as a healthy food. Food or health food preparations are not particularly limited. For example, it can be made into tablets, drinks, candies, and the like. Each food preparation may include other ingredients used in the art in addition to the homogenous polysaccharide. The other ingredients can be selected by those skilled in the art considering the specific formulation or purpose used.

In some embodiments, the composition is a food or health product including sports drinks, protein powder, a snack bar, and the like.

In another aspect, the present disclosure provides a method of making the composition or the homogenous polysaccharide as described herein. Such a method may include preparing or isolating the homogenous polysaccharide. The method may further comprise mixing the excipient and the homogenous polysaccharide.

The features and effects of the present invention will be explained through Preparation Examples and Test Examples. However, the following preparation examples and test examples are for illustration only, and do not limit the scope of the present invention.

### Examples

### 1. Material Preparation

1-1. Preparation of Lycium barbarum Extract:

Lycium barbarum exract (“LBE”) was prepared in the following general procedures: Lycium barbarum berries were cultivated in and obtained from Zhongning County, Yinchuan, Ningxia, China. The dried berries were soaked in double-distilled water (pH=7) at room temperature for 2 hours after being washed 3-5 times, then crushed. The soaked berries powder were added 5-8 times neutral water, mixed uniformly and decocted at a boiling temperature twice, with a period of time for decocting of 2.0 h and 1.5 h, respectively. The combined concentrated decoctions were filtered by a hollow fiber membrane. The above filtrates were merged and evaporated under a vacuum at 30-55° C. to remove water and obtain the concentrate. The resulting concentrate was lyophilized into a powder and stored in a desiccator, and it was used for the following experiments at suitable concentration.

In the experiments, an exemplary resulting Lycium barbarum extract was used. Such exemplary extract is an aqueous extract solution, and has a concentration of 0.4 g/mL (the equivalent mass of dry powder or dry weight of the extract/the volume of the extract). This concentration is for illustration only. The aqueous extract may be adjusted to have any suitable concentration, for example, in a range of from about 0.1 g/mL to 5 g/mL (the dry weight of the exemplary extract/the volume of extract). Such an aqueous extract may be diluted for administration in some embodiments. The aqueous extract may be further diluted for experiments with cells.

The Lycium barbarum extract (LBE) used in the present disclosure include mainly water-soluble Lycium barbarum polysaccharides, Lycium barbarum flavonoids, carotenoids, polyphenols, and pigment. In the exemplary LBE used, polysaccharide is present in a range of from about 50.0 wt. % to about 70.0 wt. % (e.g., about 54%-56% or 54%), Lycium barbarum flavone is a range of from about 0.1 wt. % to about 5.0 wt. %, carotenoid is a range of from about 0.1 wt. % to about 3.0 wt. %, polyphenol is a range of from about 0.1 wt. % to about 8.0 wt. %, and Lycium barbarum pigment is a range of from about 0.1 wt. % to about 8.0 wt. %, based on the total equivalent weight of the extract in dry powder form. LBE was in the powder form and can be dissolved in water or saline. The LBE having the same composition was used for comparison.

1-2. Isolation of L. barbarum Polysaccharides and Homogenous Polysaccharides:

L. barbarum polysaccharides (“LBP”) and homogenous polysaccharide such as an example labelled as LBP1C-2 as described herein can be isolated from the LBE or from L. barbarum berries, for example, through the following exemplary method used.

Such a method for extracting polysaccharides involves: crushing dried berries, adding 15-30 times deionized water, mixing uniformly, adding 3 wt. % cellulase, 1 wt. % amylase and 0.5 wt. % papain, performing extraction at 55-60 degrees C. for 1 hour, increasing the temperature to inactivate the enzyme, centrifuging, concentrating the obtained filtrate, dialyzing, performing reconcentration, adding 5 times 95% ethanol, centrifuging to obtain precipitates, alternately washing the precipitates using absolute ethyl alcohol and acetone for 3 times, and performing vacuum drying to obtain crude polysaccharides (LBP). Such a method further includes: adding 10-15 times water to the crude polysaccharides for dissolution, centrifuging, collecting supernatant, performing fractional purification using diethylaminoethyl cellulose (DEAE) anion exchange column, sequentially eluting using water, 0.05 M, 0.1 M, and 0.2 M sodium chloride, respectively, and collecting 0.2 M sodium chloride eluted fraction, concentrating, dialyzing and freeze-drying to obtain preliminarily purified Lycium barbarum polysaccharide (LBP1C), dissolving the obtained polysaccharide LBP1C in 0.2 M sodium chloride, centrifuging, collecting the supernatant, eluting using Sephacryl-300 (RTM: Poly((allyl dextran)-co-N,N′-methylenebisacrylamide)) column, collecting the eluted fraction, concentrating, and performing dialysis and freeze-drying treatment to obtain polysaccharide LBP1C-2, which is a homogeneous polysaccharide.

LBP1C-2 has the compositions and structures described herein.

### 1-3. Molecular Weight Detection of LBP1C-2:

The molecular weight and the homogeneity of LBP1C-2 were measured by high-performance gel-permeation chromatography (HP-GPC). Only one symmetrical peak appeared in the HP-GPC result plot. For the LBP1C-2 sample used, the weight-averaged molecular weight (Mw), the number averaged molecular weight (Mn) and the polydispersity (PD) index were estimated to be 13,181 Da, 10,750 Da, and 1.22, respectively, in reference to the molecular weight-known dextran standards used in the HP-GPC measurement.

### 1-4. Preparation of Sulfated Polysaccharide Derivative of LBP1C-2 and Measurement of the Degree of Sulfate Substitution (DS)

The homogenous polysaccharide was sulfated according to the chlorosulfonic acid-pyridine method. LBP1C-2 (50 mg) was dissolved in 2.5 mL of dried formamide. 1.5 mL of sulfation reagent made from chlorosulfonic acid and pyridine (3:1, v/v) was added under ice bathing. The mixture was then stirred at 40° C. for 4 h, cooled and neutralized with 5 M NaOH. The solution was dialyzed first against saturated NaHCO₃, then against distilled water. The retentate was lyophilized to give the sulfated derivative S-LBP1C-2. The degree of sulfation was calculated according to chlorosulfonic-pyridine method, using Equation (1) as follows:

$$D S = \frac{1.62 \times \% S}{32 - 1.02 \times \% S} , \qquad ( 1 )$$

where DS is the degree of sulfate substitution, and % S is the percentage sulfur content.

The degree of sulfate substitution (DS) of S-LBP1C-2 was calculated to be 0.74 based on the sulfur content (10%), in reference to sulfate content standard curve detecting by chlorosulfonic-pyridine method.

### 2. Biological Experiments:

### 2-1. Animals and Groups

Healthy wild-type (WT) adult C57BL/6J mice (male, 2 months old), and 14-month-old naturally aging male C57BL/6J mice were housed in an SPF barrier facility. Mice were randomly assigned to adult groups (15 per group) and aging groups (6 per group). The adult and aging mice were divided into 4 groups: a vehicle group, an LBE group, an LBP group, and an LBP1C-2 group. The vehicle group mice received distilled water (10 mL/kg) by oral gavage, while three empirically determined doses of LBE (40 mg/kg), LBP (40 mg/kg) or LBP1C-2 (40 mg/kg) were administered by oral gavage once daily for 4 months. All animal experiments comply with the ARRIVE guidelines and the U.K. Animals (Scientific Procedures) Act, 1986 and associated guidelines.

### 2-2. Cell Culture

hMSCs were purchased from ScienCell Research Laboratories. The growth culture medium used in this study was α-minimum essential medium (α-MEM; Gibco, 12571063) supplemented with 10% fetal bovine serum (FBS; Gibco, 16140071) and 1% penicillin-streptomycin (HyClone, SH40003-12). For the differentiation culture medium, α-MEM was supplemented with 10% FBS, 1-ascorbic acid (50 μg/ml; Sigma, A8960-5G), 0.1 μM dexamethasone (Sigma, D8893-1 MG), 10 mM β-glycerophosphate (Sigma, G9422-10G), and 1% penicillin-streptomycin (HyClone, SH40003-12). The medium was refreshed every 2 days. The cell cultures were then incubated in a humidified environment with 5% CO₂ at 37° C.

### 2-3. Evaluation of Serum Markers of Bone Formation

Blood samples were taken from the fundus of mice after treatment following anaesthesia, and the serum was obtained after centrifugation (2000×g, 20 min, 4° C.). Serum activities of Osteocalcin and PINP, which are both markers of bone formation, were measured using an Osteocalcin ELISA Kit and a PINP ELISA Kit, respectively, according to the manufacturer's protocols.

### 2-4. Analysis of Bone by Micro-CT

A micro-computed tomography (micro-CT) imaging system (Inveon MM system, Siemens, Munich, Germany) was employed to perform the CT scan and trabecular morphometric analysis according to the manufacturer's procedure. The measured trabecular metric parameters included BMD, BV/TV, Tb.N, Tb.Th, and Tb.Sp. Briefly, the right femur samples were scanned at an effective pixel size of 8.89 μm, a voltage of 60 kV, a current of 220 μA, and an exposure time of 1500 ms in each of the 360 rotational steps, in vivo and ex vivo. The images consisted of 1536 slices and had a voxel size of 8.89 μm in all three axes. Three-dimensional (3D) visualization images were reconstructed by 2D images, and the parameters were calculated using Inveon Research Workplace (Siemens). The trabecular region of interest (ROI) of the femur was 1-2 mm below the distal growth plate.

### 2-5. Three-Point Bending Test

A small-animal bone strength testing instrument (Instron 4302, Instron, Norwood, Mass) was used to perform the three-point bending test. Immediately after dissection, the fresh humerus was examined using the three-point bending test. Two end support points and one central loading point were used for the three-point bending test. The biomechanical measurement data were collected from the load-deformation curves.

### 2-6. SPR-Based Binding Assay

The affinity of the binding of LBP1C-2 to the proteins was measured using a BIACORE T200 (GE Healthcare, Stockholm, Sweden). The BMPRIA, BMPRII and Noggin protein was immobilized on a CM5 sensor chip by the ammine coupling method. For interaction measurements, different concentrations of LBP1C-2 were injected into the chips. All procedures were conducted in HBS-EP running buffer (pH 7.4) containing 0.01 M HEPES, 0.15 M NaCl, 3 mM EDTA and 0.005% surfactant P20. Kinetic parameters were determined by fitting the data using a 1:1 binding model with Biacore T200 Evaluation Software, version 1.0.

### 2-7. Cellular Thermal Shift Assay (CETSA)

The CETSA was carried out based on a previously described protocol (Li et al., 2017). hMSCs were cultured for 96 h in a medium containing vehicle or 4 μM LBP1C-2. Then the cells were collected. RIPA lysis buffer was added, frozen and thawed 3 times using liquid nitrogen, and centrifuged at 12,000 g for 10 min at 4° C. The supernatant of the vehicle-treated group was divided into two equal parts. One part of the vehicle-treated group was treated with ddH2O2 for 30 min, and the other part of the vehicle-treated group and the 8 μM LBP1C-2 treated group were treated with vehicle for 30 min. Soluble proteins were collected by centrifugation at 12,000 g for 15 min at 4° C. and then detected by Western blotting.

### 2-8. Quantitative Real-Time PCR Assay (RT-PCR)

Femur tissues or cells were harvested, and total RNA was extracted using TRIzol reagent (87803, Invitrogen, USA) according to the manufacturer's protocol (Invitrogen). mRNA levels were measured by qRT-PCR using a 7500 real-time PCR system (Applied Biosystems), as described previously. The change in mRNA expression in rats in each treatment group was assessed by the 2-ΔΔCq method.

### 2-9. Western Blot Analysis

To prepare the femur tissues or cells for analysis, they were lysed using RIPA buffer. The RIPA buffer composition included 50 mM Tris (pH 7.4), 150 mM NaCl, 1% Nonidet P-40, 0.5% DOC, 0.1% SDS, 5 mM EDTA, and a protease inhibitor cocktail solution (Roche). Next, 40 mg of protein from each sample was separated using sodium dodecyl sulfate-polyacrylamide gel electrophoresis (SDS-PAGE) and transferred to an NC membrane. The membranes were then blocked in a solution of 5% skim milk powder in TBST (10 mM Tris, 150 mM NaCl, 0.1% Tween-20; pH 7.4) for 1 hour. Following the blocking step, the membranes were incubated overnight at 4° C. with the primary antibodies. The primary antibodies used in this study included anti-p-Smad1/5/8 (1:1000), anti-Smad1/5/8 (1:1000) and anti-GAPDH (1:2000). Afterward, the membranes were washed with TBST and incubated at room temperature for 1 hour with a secondary antibody conjugated to horseradish peroxidase (HRP) at a 1:2000 dilution (Zhong-shan Jin-qiao Corp., Beijing, China). The membranes were washed three times with TBST. To visualize the protein signals, each membrane was treated with an ECL solution (Thermo Scientific, Waltham, MA, USA), and the signals were detected using a Molecular Imager ChemiDoc XRS+ (Bio-Rad).

### 2-10. ALP Staining and ALP Assay

ALP staining was monitored using a Vector Blue substrate kit (procedure number SK-5300, Vector Laboratories). According to the protocol, hMSCs were plated at a density of 1×10⁵ cells per well in 24-well plates and expanded in a growth medium for 3 days. At day 7, hMSCs were incubated with the substrate working solution for 30 min. During the whole procedure, the cells were protected from light. The ALP assay was carried out based on a previously described protocol.

### 2-11. Alizarin Red S Staining

hMSCs were plated at a density of 1×10⁵ cells per well in 24-well plates and expanded them in a growth medium for 3 days. Osteoblast differentiation was induced by culturing in a differentiation culture medium. At day 14 after inducing osteoblast differentiation, cells were fixed with 4% paraformaldehyde for 15 min and 0.1% Triton X-100 for 10 min and then stained with 2% (w/v) Alizarin Red S solution (pH 4.2) for 10 min using the standard protocol.

### 3. Results

In the Experiments described below, the LBE, LBP and LBP1C-2 solutions were freshly prepared by dissolving the powder in distilled water. The compositions were administrated once daily. The vehicle control group mice received distilled water by oral gavage, while three empirically determined doses of LBE (40 mg/kg), LBP (40 mg/kg) or LBP1C-2 (40 mg/kg) were administered by oral gavage once daily for 4 months to mice in the treatment groups. The dose of the effective amount of LBP1C-2 (by the amount of the homogenous polysaccharide as described herein) was 40 mg/Kg based on a total daily weight of the homogeneous polysaccharide/a body weight of the subject on daily basis. The doses of LBE and LBP (40 mg/kg) are a total daily weight of LBE or LBP/a body weight of the subject.

### 3-1. LBE, LBP and LBP1C-2 Treatment Increases Bone Mass and Stability.

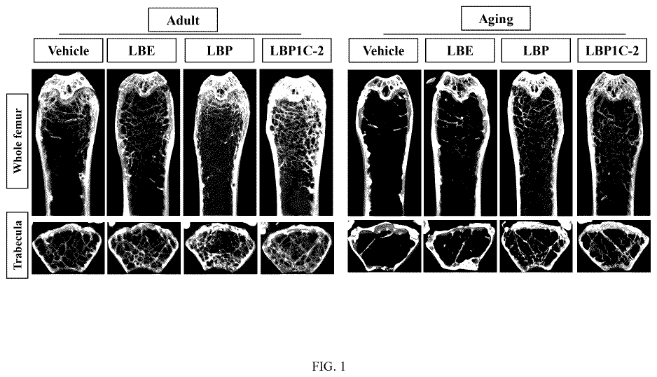

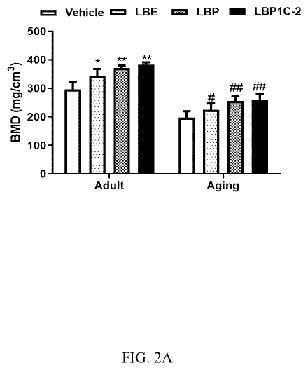

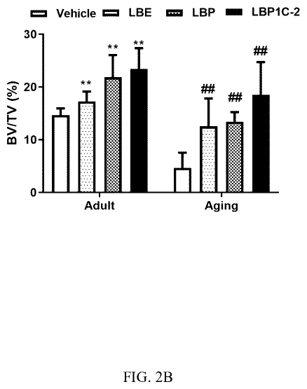

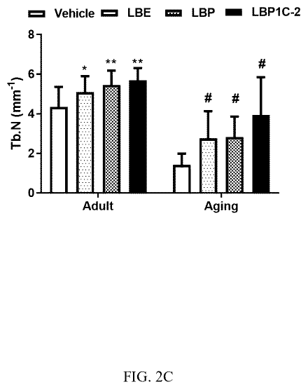

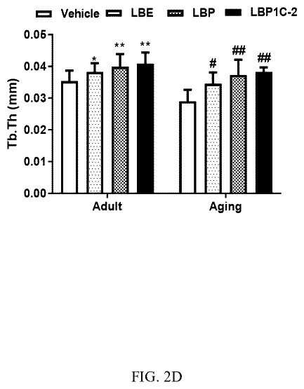

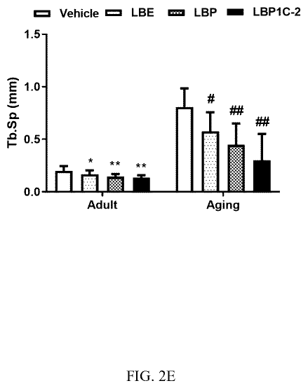

Aging is correlated with low bone mass and associated with deterioration of the bone microarchitecture. Therefore, tests was performed to study whether the LBE, LBP and LBP1C-2 affects trabecular structural parameters in mice by micro-computed tomography (μCT). Using μCT, bone morphometric data obtained from vehicle and treatment mouse femurs were quantified. When compared with the adult and aging vehicle group, LBE, LBP and LBP1C-2 treatment showed better trabecular structural parameters (FIG. 1). When compared with the adult and aging vehicle group, LBE, LBP and LBP1C-2 treatment appeared to afford improvement of the aging-induced effect on the trabecular bone as LBE and LBP-treated mice showed higher BMD (bone mineral density) (FIG. 2A), increased trabecular bone volume to total volume (BV/TV) ratio (FIG. 2B), increased trabecular number (Tb.N) (FIG. 2C), increased trabecular thickness (Tb.Th) (FIG. 2D) and decreased trabecular separation (Tb.Sp) (FIG. 2E). *p<0.05, **p<0.01 (compared to the adult vehicle group); #p<0.05, ##p<0.01 (compared to the aging vehicle group).

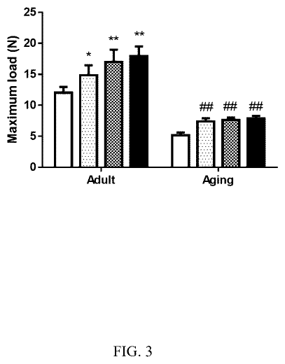

Next, it was investigated whether LBE, LBP and LBP1C-2 regulates the biomechanics of bone. The data showed that the Max load of LBE, LBP and LBP1C-2 treated mice was remarkably increased compared to adult and aging vehicle group (FIG. 3).

### 3-2. LBE, LBP and LBP1C-2 Treatment Positively Influences Bone Formation

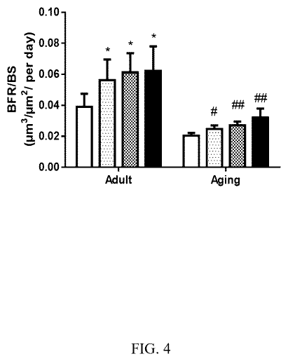

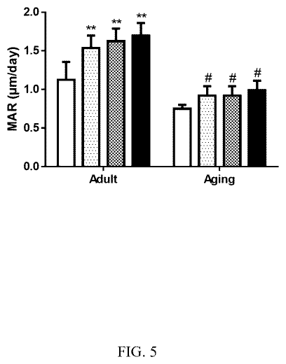

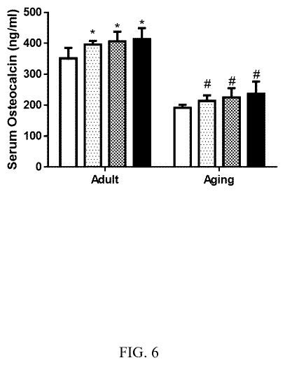

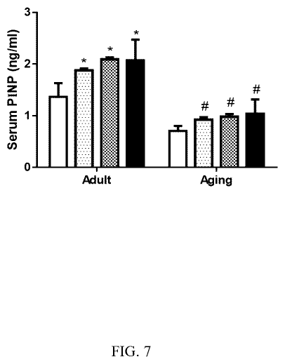

Analyses were performed to study bone formation rate and osteoid in the femur body. The bone formation rate (BFR) represents the amount of new bone formed per unit of time. It is typically measured by quantifying the amount of mineralized bone matrix or osteoid (unmineralized bone) formed within a specific bone surface area over a given period. The mineral apposition rate (MAR) focuses specifically on the rate of mineralization of the newly formed bone matrix. It measures the speed at which mineral crystals, primarily hydroxyapatite, are deposited onto the osteoid surface during bone formation. The bone formation rate (BFR, FIG. 4) and mineral apposition rate (MAR, FIG. 5) was remarkably increased in LBE, LBP and LBP1C-2 treated mice by the calcein double labeling assay. Significantly increased in serum levels Osteocalcin, a marker of osteoblastic (FIG. 6) activity and PINP, a bone formation marker (FIG. 7) when compared with the vehicle group. The effect of LBP1C-2 was more evident. Taken together, these results suggest that LBE, LBP and LBP1C-2 supplement regulates bone mass by modulating bone formation and osteoblast mineralization. *p<0.05, **p<0.01 (compared to the adult vehicle group); #p<0.05, ##p<0.01 (compared to the aging vehicle group)

### 3-3. LBE, LBP and LBP1C-2 Enhance Osteoblast Proliferation, Differentiation, and Mineralization.

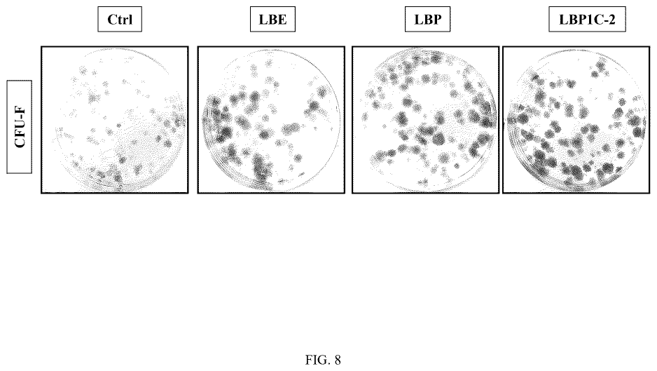

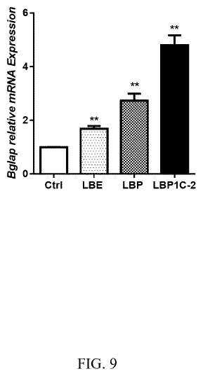

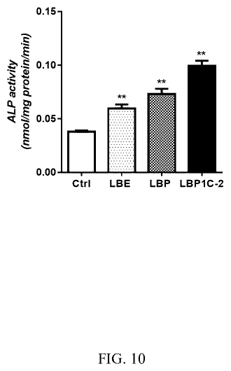

To measure whether LBE, LBP and LBP1C-2 affects human mesenchymal stem cells (hMSCs) differentiation to osteoblasts and gain additional insights into the specific compound. hMSCs were used to analyze osteoblast proliferation, differentiation and mineralization. The results showed that the proliferation of hMSCs were promoted by LBE, LBP and LBP1C-2 of CFU-F (fibroblast colonyforming unit) colonies (FIG. 8). Similarly, osteoblast differentiation and mineralization were markedly accelerated by LBE, LBP and LBP1C-2. Furthermore, two osteoblast differentiation markers, Bglap expression (FIG. 9) and ALP enzyme activity (FIG. 10), were increased in LBE, LBP and LBP1C-2 treatment cells. All of the results indicate that LBE, LBP and LBP1C-2 treatment enhances hMSCs differentiation and mineralization to osteoblasts.

### 3-4. LBP1C-2 Stimulates Bone Formation and has Little Effect on Bone Resorption

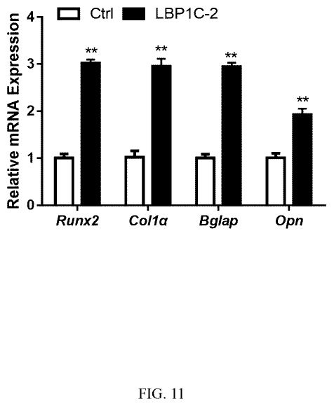

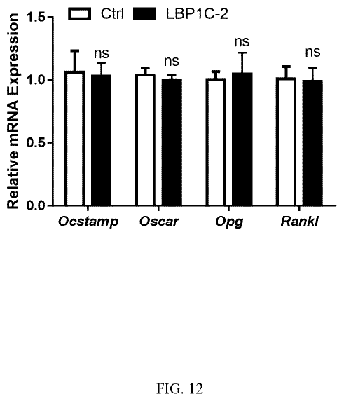

To elucidate the mode of action of the LBP1C-2, expression of bone formation and bone resorption genes were analyzed by RT-PCR. The mRNA expression of osteogenic differentiation mediators, namely Col1a, Runx2, Bglap and Opn were significantly up-regulated occurred in LBP1C-2 treated cells (FIG. 11). To further confirm the effect of LBP1C-2on bone resorption, the mRNA expression of osteoclast genesis markers includes Ocstamp, Oscar, Opg and Rankl were measured in femur and there were no significant differences (FIG. 12). These results suggest that LBP1C-2 increases bone formation and does not affect bone resorption.

### 3-5. LBP1C-2 Enhances Bone Formation Depending on BMPRIA and BMPRII.

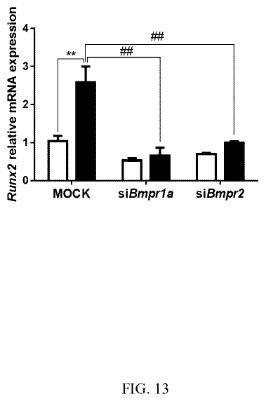

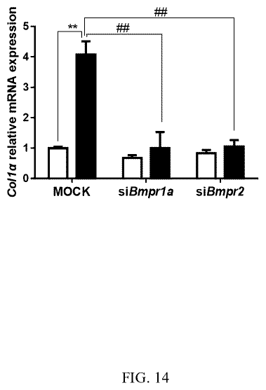

Since BMPR is essential receptor for osteoblastic differentiation, it was elucidated whether its expression was correlated with osteoblastic differentiation induced by LBP1C-2. A different amount BMPR-siRNA was used to confirm the specific inhibitory effect targeting to BMPRIA and BMPRII. The Quantitative real-time PCR analysis indicated the expression levels of downstream Col1a, Runx2 and Bglap. Moreover, BMPRIA and BMPRII silencing led to the downregulation of Runx2 (FIG. 13), Col1α (FIG. 14), and Bglap (FIG. 15), indicating osteoblastic differentiation and mineralization induced by LBP1C-2 treatment was inhibited with the downregulation of BMPRIA and BMPRII. Thus, the results indicate that BMPRIA and BMPRII regulated osteoblastic differentiation and mineralization induced by LBP1C-2.

3-6. LBP1C-2 Directly Binds BMPRIA, BMPRII and Substantially Activates them.

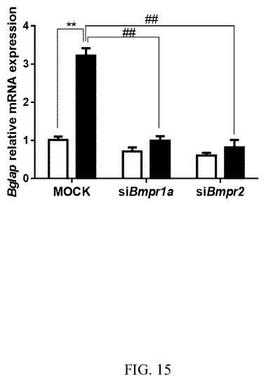

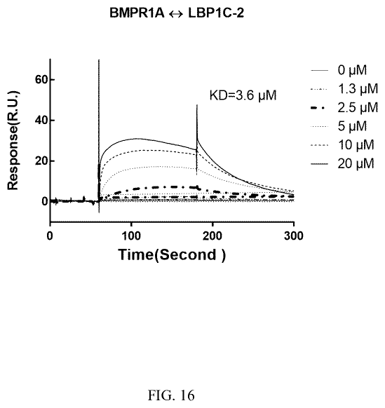

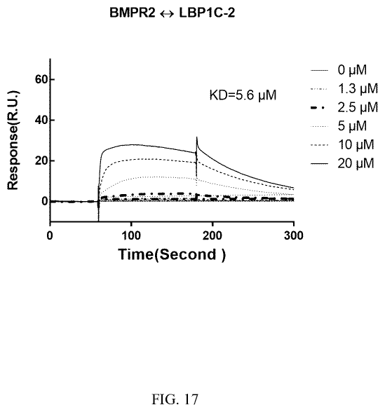

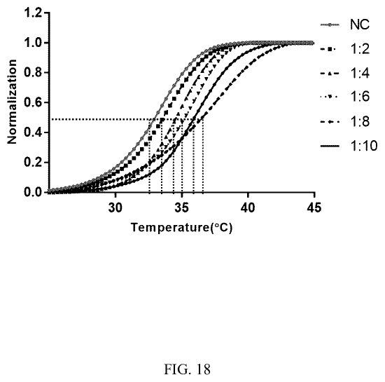

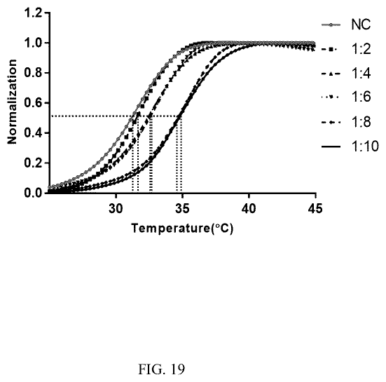

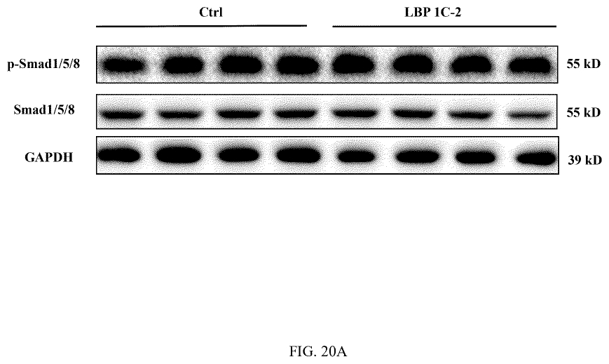

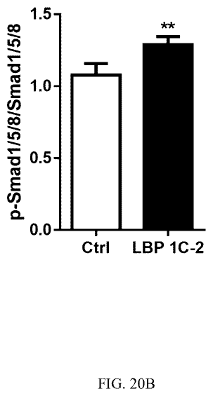

To validate whether LBP1C-2 directly interacted with BMPR, to address this question, the interactions were measured by SPR. The results showed that the interactions were dose-dependent. The equilibrium dissociation constant (Kd) between LBP1C-2 and BMPRIA was approximately 3.6 μmol/L (FIG. 16) and the Kd of BMPRII was 5.6 μmol/L (FIG. 17). The direct interaction between LBP1C-2 and BMPR was further confirmed by a fluorescence based thermal shift assay; LBP1C-2 dose dependently shifted the melting temperature (Tm) of BMPRIA (FIG. 18) and BMPRII by more than 4° C. before reaching a plateau (FIG. 19). BMP/Smads signaling is the key pathways to regulate BMSC differentiation into osteoblasts. To validate whether LBP1C-2 regulates osteoblast differentiation by the BMP/Smads signaling pathway in hMSCs, phosphorylation levels of Smad1/5/8 were measured after 14 days of differentiation from hMSCs to osteoblasts. Phosphorylation levels of Smad1/5/8 were significantly increased in hMSCs by LBP1C-2 treatment by Western blot and quantification analyses (FIGS. 20A-20B). These data demonstrate that LBP1C-2 directly binds BMPR and activates BMPRIA and BMPRII, markedly increased the downstream the phosphorylation of Smad1/5/8. Therefore, LBP1C-2 through binding BMPRIA and BMPRII, up-regulating BMP/Smads signaling pathway to enhance bone formation.

3-7. LBP1C-2 Blocks Noggin Interaction with BMPs.

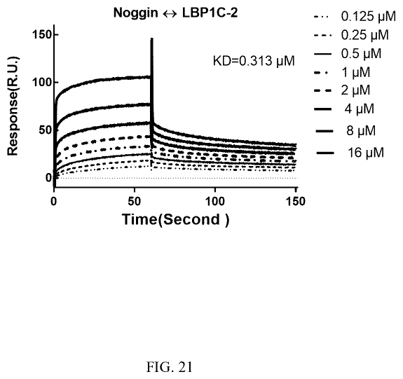

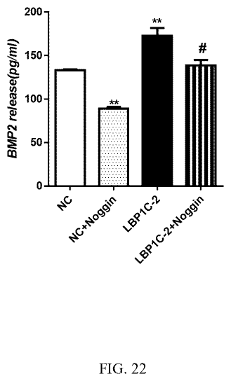

It is established that LBP1C-2 directly activates BMPRIA and BMPRII, activation of the BMPRIA and BMPRII ultimately promotes phosphorylation of the downstream effector Smad1/5/8 to enhance osteogenic differentiation and mineralization. Noggin is known for its inhibitory effects on BMP signaling. Because noggin interaction with BMPs inhibits osteogenic differentiation, the inventors investigated whether LBP1C-2 binding to noggin also activated this pathway. As shown in FIG. 21, treatment with LBP1C-2 to detect how LBP1C-2 affects noggin, the interaction of LBP1C-2 and noggin were determined by SPR analysis. The result showed that LBP1C-2 strongly bound to noggin, with an equilibrium dissociation constant (Kd) of 0.313 μM. Furthermore, whether this bound inhibited noggin interaction with BMP2, the concentration of BMP2 in the supernatants of LBP1C-2 treatment hMSCs was measured. The result suggested that under noggin condition a spot level of BMP2 and LBP1C-2 condition significant amounts of BMP-2 were detected in cell supernatants. LBP 1C-2 induced up-regulation of the BMP-2 level was also inhibited by noggin. These data suggested that LBP1C-2 interacted with noggin and inhibited noggin bound to BMP2 (FIG. 22).

Age-related bone loss appears to be same in men and women, and fractures resulting from osteoporosis are a major cause of morbidity and mortality in older people. In the present study, the inventors firstly demonstrated the protective effect of L. barbarum on the bone in a natural aging model. LBE, LBD and LBP1C-2 supplement as described herein successfully ameliorated age-induced osteoporosis in mice by improving overall bone microstructures. Further, the main active substance was LBP1C-2 was identified. The mechanism study showed that LBE, LBD and LBP1C-2 modulated bone formation rather than bone resorption. LBP1C-2 as the main component of Lycium barbarum could directly bind BMPRIA, BMPRII to promote phosphorylation of the downstream effector Smads to enhance osteogenic differentiation and mineralization. Otherwise, LBP1C-2 was also directly bound to noggin and inhibited noggin interaction with BMPs, and this binding further upregulated BMP/Smads signaling pathway to increase bone formation. These results indicated that these composition as described herein have the mimetic effects of BMPs in osteogenic function potential to treat age-related bone loss as a dietary supplement or pharmaceutical composition.

Naturally aging model is the most appropriate tool to best mimic age-related bone loss although requiring a long time, the inventors' study of L. barbarum in naturally aging mouse model owns beneficial advantage. LBE, LBD and LBP1C-2 supplement as described herein successfully ameliorated bone loss and age-related bone loss in adult mice and aged mice.

BMPR silencing led to Col1α1, Runx2 and Bglap downregulated in LBP1C-2 treatment pre-osteoblasts. Furthermore, SPR, Thermal shift assay, and CETSA were used to validate whether LBP1C-2 directly interacted with BMPR. The inventors found that LBP1C-2 can directly bind the BMPRIA, BMPRII and the downstream effector Smad1/5/8 phosphorylation levels were significantly increased. On the other hand, the inventors firstly found that LBP1C-2 not only binds the BMPRIA and BMPRII, but also directly binds noggin stronger, and this binding blocked noggin interaction with BMPs. These two interactions resulting in osteogenesis differentiation suggested that LBP1C-2 mimic ligand of BMPR to regulate ossification via BMP/Smads signaling pathway.

While currently there are no specific drugs available for the treatment of age-related osteoporosis, the routine used drugs have certain limitations and long-term effect is still unclear. Clinically, osteoporosis treatment options are mainly divided into the following strategies, antiresorptive drugs (Denosumab, Odanacatib, Saracatinib, etc.) are commonly used to treat osteoporosis, but inhibition of bone resorption also interferes with bone remodeling and inhibit bone formation. Serious side effects of these drugs designed to combat osteoporosis are not uncommon, such as osteonecrosis of the jaw and atypical bone fracture. Teriparatide, a recombinant parathyroid hormone, is currently the main anabolic therapeutic option for senile osteoporosis by daily subcutaneous injections with appropriate dosage. Against this backdrop, natural products, which has the potential of milder side effects on bone health, could present a better alternative or approach to the management of osteoporosis. Lycium barbarum is used as a traditional Chinese medicine or healthy food for treatment of age-related diseases. In the present disclosure, it has demonstrated that a pure polysaccharide LBP1C-2, which is isolated from Lycium barbarum supplement, for long term treatment (4 months treatment in mice) resulted in a remarkable increase in bone mass and bone strength of adult and aging mice, and the adverse effects were not found. Therefore, LBP 1C-2 may be a potentially effective and safe compound for adjuvant drug development in pathological and non-pathological conditions.

In summary, the study in the present disclosure has demonstrated that LBP1C-2, a pure polysaccharide from Lycium barbarum can be used for treating or preventing age-related bone loss. Age-related bone loss is an emerging medical and socioeconomic threat due to its high prevalence, high disability rate and high fatality rate. Lycium barbarum has beneficial health effects as a traditional Chinese medicine and also an edible food. However, whether Lycium barbarum regulates age-related bone loss and what is the major active substance is unknown. In the present disclosure, the results have shown the effects of three types of Lycium barbarum extracts: Lycium barbarum water extracts (LBE), Lycium barbarum polysaccharides (LBP, isolated from LBE) and a pure polysaccharide LBP1C-2 (isolated from LBP) as described herein on treating or preventing bone loss. LBE, LBP and LBP1C-2 supplement resulted in a remarkable increase in bone mass and bone strength in adult mice and aging mice. Osteoblast proliferation, differentiation and ossification were also promoted by LBE, LBP and LBP1C-2 in hMSCs and also in treated mice (in vivo). Furthermore from the mechanism, the main polysaccharide ingredient LBP1C-2 directly binds BMP receptors, BMPRIA and BMPRII, the activation of the BMPRIA and BMPRII ultimately promotes phosphorylation of the downstream effector Smads to enhance osteogenic differentiation and mineralization. Otherwise, noggin is known as a potent inhibitor of BMPs, and LBP1C-2 was also directly bound to noggin and inhibited noggin interaction with BMPs. This binding further upregulated BMP/Smads signaling pathway to increase bone formation. Taken together, the results indicate that the compositions from Lycium barbarum prevent from aging induced bone loss, and the main polysaccharide ingredient LBP1C-2 regulates age-related bone loss through BMPRIA, BMPRII and noggin of BMP/Smads signaling pathway. This study also might provide a basis for further study of LBE, LBP and LBP1C-2 as functional foods to interfere with age-related osteoporosis.

### 3-8. Results of Sulfate-Derivative (S-LBP1C-2) of LBP1C-2

Sulfate-derivative of LBP1C-2 was prepared according to the procedures described above. The derivative is called S-LBP1C-2.

To investigate the potential of sulfated LBP1C-2 (S-LBP1C-2) in promoting osteoblast differentiation and mineralization, human mesenchymal stem cells (hMSCs) were employed to assess the impact of S-LBP1C-2 on the expression of early osteogenic markers involved in bone formation. RUNX2 and SP7 (also known as Osterix) are two key transcription factors that play crucial roles in bone formation and osteoblast differentiation. RUNX2 is considered the master regulator of osteoblast differentiation. It is essential for the commitment of mesenchymal stem cells to the osteoblast lineage and the subsequent maturation of osteoblasts. RUNX2 regulates the expression of various genes involved in osteoblast differentiation, extracellular matrix formation, and mineralization. SP7, on the other hand, acts downstream of RUNX2 and is considered a marker of mature osteoblasts. It is a transcription factor that is primarily expressed in osteoblasts and is responsible for their differentiation and function.

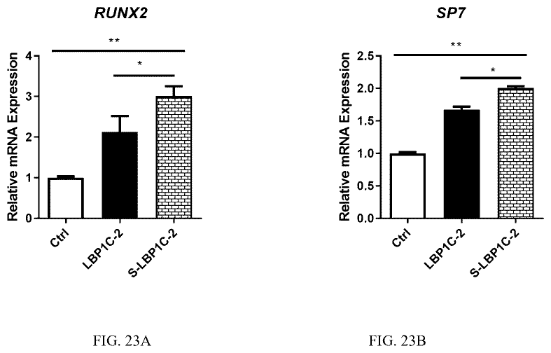

Initially, the levels of the primary osteogenic markers, RUNX2 and SP7, were analyzed using qPCR after treating hMSCs for a duration of 7 days. The results demonstrated that S-LBP1C-2 increased the mRNA levels of RUNX2 and SP7 (FIGS. 23A and 23B, respectively). This finding suggests that S-LBP1C-2 possesses the ability to promote osteoblast differentiation. Furthermore, when comparing at an equivalent concentration of 4 μmol, it was observed that LBP1C-2 upregulated RUNX2 by 2.13-fold, while S-LBP1C-2 upregulated RUNX2 by 3.01-fold. Similarly, LBP1C-2 upregulated SP7 by 1.68-fold, whereas S-LBP1C-2 upregulated SP7 by 2.01-fold. Thus, the effect of S-LBP1C-2 was found to be superior to that of LBP1C-2.

BALP staining, also known as alkaline phosphatase staining, is a commonly used technique to assess the activity of alkaline phosphatase (ALP) enzyme in cells undergoing osteoblast differentiation. ALP is an enzyme predominantly expressed by osteoblasts and is involved in various aspects of bone formation. During the process of osteoblast differentiation, the expression and activity of ALP increase. ALP staining allows researchers to visualize and quantify ALP activity, providing an indirect measure of osteoblast differentiation and maturation.

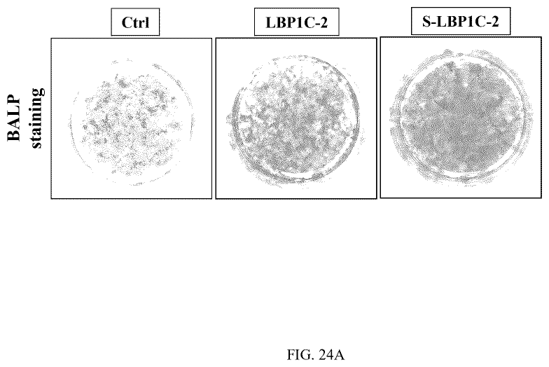

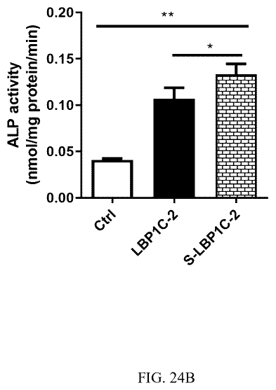

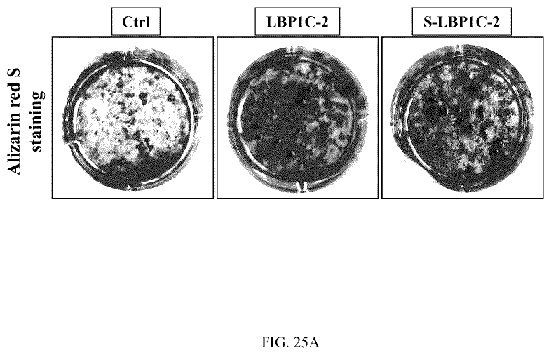

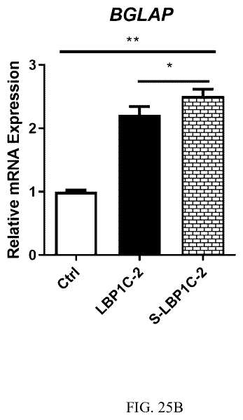

Subsequently, the impact of S-LBP1C-2 on ALP staining (FIG. 24A) and ALP activity (FIG. 24B) was evaluated. S-LBP1C-2 was observed to enhance both ALP secretion and ALP activity. The promotion of ALP secretion and activity by S-LBP1C-2 exceeded that of LBP1C-2, indicating that S-LBP1C-2 possesses a more potent capacity to facilitate osteoblast differentiation. Alizarin Red S staining is a widely used technique to detect and quantify calcium mineralization in bone and mineralized tissues. It is commonly employed to assess the extent of mineralized matrix deposition by osteoblasts during bone formation. Additionally, the effect of S-LBP1C-2 on alizarin red S staining (FIG. 25A) and the expression level of osteocalcin in hMSCs was assessed (FIG. 25B). The results of alizarin red S staining demonstrated that S-LBP1C-2 promotes osteogenic mineralization. BGLAP, also known as osteocalcin, is a protein that plays an important role in bone formation and mineralization. It is primarily synthesized and secreted by osteoblasts, the cells responsible for bone formation. BGLAP is considered a marker of osteoblast activity.

Moreover, the mRNA level of BGLAP, a gene encoding osteocalcin, was increased by S-LBP1C-2 when hMSCs were treated for 21 days. Similar to the previous findings, at an equivalent concentration of 4 μmol, it was observed that LBP1C-2 upregulated BGLAP by 2.21-fold, while S-LBP1C-2 upregulated BGLAP by 2.51-fold, the effect of S-LBP1C-2 on BGLAP expression was superior to that of LBP1C-2.

### 4. Experimental Samples and Control Samples:

Four homogenous polysaccharides were isolated from the L. barbarum extract. Among the four homogenous polysaccharides, only LBP1C-2 shows the functions as described herein. Among the four homogeneous polysaccharides, the sugar compositions and the structures of three homogeneous polysaccharides including LBP1A1-1, LBP1B-S-2, and LBP1C-2 were analyzed.

Referring to Scheme 1, LBP1A1-1 consists of Rhamnose (Rha), Arabinose (Ara), Glucose (Glc) and Galactose (Gal), the molar ratio was 1.2:47.8:1.4:49.8. Structural analysis showed that LBP1A1-1 is mainly composed of 1, 4-α-Glc, 1,3-β-Gal and 1, 6-β-Gal. Its branches mainly include Terminal (T)-β-Rha, T-β-Gal, T-α-Ara, T-β-Ara and 1, 5-α-Ara. The branches are linked to the C-6 position of the main chain sugar 1, 3-β-Gal residue and the C-3 position of 1, 6-β-Gal.

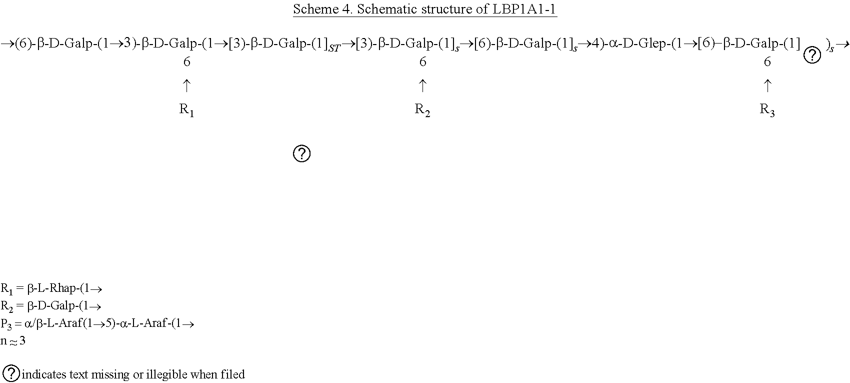

Referring to Scheme 5, LBP1B-S-2 consists of Rha, GlcA (Glucuronic acid), Gal and Ara with a molar ratio of 3.13:3.95:39.37:53.55. Structural analysis showed that LBP1B-S-2 is mainly composed of 1, 3-β-Gal, 1,6-β-Gal and its branches mainly include 1, 4-β-GlcA, T-β-Rha, T-β-Gal, T-α-Ara, T-β-Ara, 1,5-α-Ara and part of 1, 6-β-Gal. The branches are linked to the C-6 position of the main chain sugar residue 1, 3-β-Gal and the C-3 position of 1, 6-β-Gal.

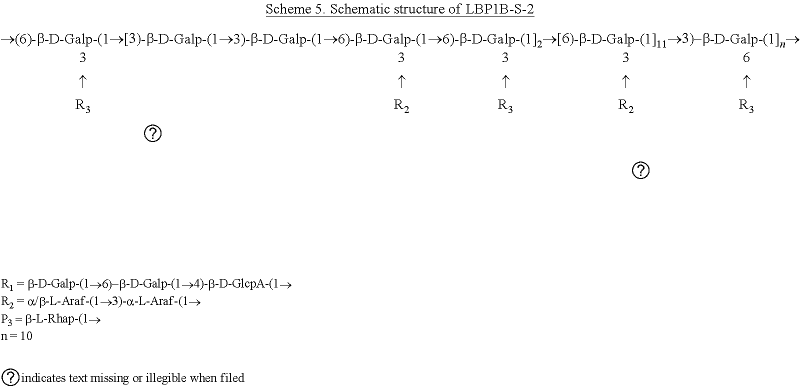

Among the four homogenous polysaccharides with distinct structures, LBP1C-2 demonstrated the most robust functions and activities. LBP1C-2, its derivative such as sulfate derivative, and a composition comprising LBP1C-2 or a derivative thereof are the preferred compositions for improving bone formation, bone mass and/or bone strength, and for treating or preventing bone loss such as age-related bone loss in a subject in need thereof in accordance with some embodiments.

Although the subject matter has been described in terms of exemplary embodiments, it is not limited thereto. Rather, the appended claims should be construed broadly, to include other variants and embodiments, which may be made by those skilled in the art.

What is claimed is:

1. A method for treating or preventing bone loss and/or improving bone formation, bone mass or strength in a subject in need thereof, comprising:
administrating an effective amount of a composition comprising a homogenous polysaccharide, or a pharmaceutically acceptable ester or salt thereof, or a pharmaceutically acceptable solvate thereof, or any combination thereof, and a pharmaceutically acceptable excipient into a subject in need thereof,
wherein the homogeneous polysaccharide consists essentially of arabinose, galactose, rhamnose, and galacturonic acid as monomer units.

2. The method of claim 1, wherein the subject is a mammal.

3. The method of claim 1, wherein the subject is a human subject, and/or the bone loss is age-related bone loss.

4. The method of claim 1, wherein the composition is orally administrated.

5. The method of claim 1, wherein the homogeneous polysaccharide has a molecular weight in a range of from about 10 kDa to about 150 kDa, and/or a ratio of monomer units of arabinose, galactose, rhamnose, and galacturonic acid in a range of from 30-70:20-60:0.1-10:0.1-10.

6. The method of claim 1, wherein the homogeneous polysaccharide has a ratio of monomer units of arabinose, galactose, rhamnose, and galacturonic acid being 49.9:33.6:8.0:8.5.

7. The method of claim 1, wherein the composition further comprises additional polysaccharide isolated from a Lycium barbarum extract, and/or the homogenous polysaccharide is at least 15% of all polysaccharides in the composition.

8. The method of claim 1, wherein the composition further comprises one or more compounds selected from the group consisting of flavone, carotenoid, polyphenol, pigment, or any combination isolated from a Lycium barbarum extract.

9. The method of claim 1, wherein the homogeneous polysaccharide is the only polysaccharide in the composition.

10. The method of claim 1, wherein the pharmaceutically acceptable ester or salt thereof is a sulfate ester derivative of the homogeneous polysaccharide.

11. The method of claim 1, wherein the excipient is a solvent, a co-solvent, a coloring agent, a preservative, an antimicrobial agent, a filler, a binder, a disintegrate, a lubricant, a surfactant, an emulsifying agent, a suspending agent, or any combination thereof.

12. The method of claim 1, wherein the dose of the effective amount of the composition is in a range of from 10 mg/Kg to 500 mg/Kg based on a weight of the homogeneous polysaccharide or the pharmaceutically acceptable ester or salt thereof/a body weight of the subject on daily basis.

13. A composition for improving bone formation, bone mass or strength and/or treating or preventing bone loss in a subject in need thereof, comprising:
an effective amount of a homogenous polysaccharide, or a pharmaceutically acceptable ester or salt thereof, or a pharmaceutically acceptable solvate thereof, or any combination thereof, and a pharmaceutically acceptable excipient,
wherein the homogeneous polysaccharide consists essentially of arabinose, galactose, rhamnose, and galacturonic acid as monomer units.

14. The composition of claim 13, wherein the pharmaceutically acceptable ester or salt thereof is a sulfate ester derivative of the homogeneous polysaccharide.

15. The composition of claim 14, wherein the sulfate ester derivative of the homogeneous polysaccharide has a degree of sulfate substitution in a range of from 0.5 to 0.9.

16. The composition of claim 13, wherein the composition is a pharmaceutical composition, a functional composition, and/or a dietary supplement.

17. The composition of claim 13, wherein the composition is an oral composition and/or is in a tablet form.

18. The composition of claim 13, wherein the excipient is a solvent, a co-solvent, a coloring agent, a preservative, an antimicrobial agent, a filler, a binder, a disintegrate, a lubricant, a surfactant, an emulsifying agent, a suspending agent, or any combination thereof.

19. The composition of claim 13, wherein the homogeneous polysaccharide or its derivative thereof has a molecular weight in a range of from about 10 kDa to about 150 kDa, and a ratio of monomer units of arabinose, galactose, rhamnose, and galacturonic acid in a range of from 30-70:20-60:0.1-10:0.1-10.

20. The composition of claim 13, wherein the homogeneous polysaccharide has a ratio of monomer units of arabinose, galactose, rhamnose, and galacturonic acid being 49.9:33.6:8.0:8.5.

21. The composition of claim 13, wherein the homogeneous polysaccharide is the only polysaccharide in the composition.

22. The composition of claim 13, wherein the composition is a pharmaceutical composition for treating age-related bone loss in a human subject.
